(19) United States
(12) Patent Application Publication
(10) Pub. No.: US 2005/0037074 A1
(43) Pub. Date: Feb. 17, 2005

(54) Delayed and sustained drug release

(76) Inventors: Richard Ross, Sheffield (GB); Amin Rostami-Hodjegan, Sheffield (GB); Geoffrey Tucker, Sheffield (GB)

Correspondence Address: CROWELL & MORING LLP;INTELLECTUAL PROPERTY GROUP, P.O. BOX 14300, WASHINGTON, DC, 20044-4300, US

(21) Appl. No.: 10/486,700

(22) PCT Filed: Aug. 8, 2002
(86) PCT No.: PCT/GB02/03631

(30) Foreign Application Priority Data
Aug. 15, 2001 (GB) 0119848.0

(51) Int. Cl. A61K 9/22; A61K 31/573

(52) U.S. Cl. 424/468; 514/179

(57) ABSTRACT

The invention relates to the controlled release of preparations of therapeutic agents, for example a steroid; formulations comprising said preparations; and the use of said formulations to treat diseases such as those diseases which would benefit from steroid treatment.

The invention relates to the controlled release of preparations of therapeutic agents, for example steroids; formulations comprising said preparations; and the use of said formulations to treat diseases which would benefit from steroid treatment.

It has long been desirable to provide drugs which can be released in a controlled manner. Controlled drug release may be viewed in two ways. Firstly, to provide a sustained drug release over a period of time so that a whole body is not flooded with the drug when administered. The drug is then cleared by the body resulting in a rapid fall in systemic levels thereby not providing adequate therapeutic effect over an entire treatment regime. A second view of controlled drug release is the situation when the delivery of the drug is desired at a specific time and in a precise manner. In this case maintaining a constant systemic level may not be desirable and indeed result is adverse side effects. Problems therefore arise when a condition requires both sustained drug release but which has to be regulated in a specific manner, for example in accordance with a circadian rhythm or menstrual cycle.

An example of a class of diseases which has both the above elements are conditions resulting from adrenal failure which result in hormonal insufficiency and diseases which may be worse at certain times of the day such as rheumatoid arthritis and asthma.

Adrenal failure occurs in approximately 1/10,000 of the population. It may be due to either primary adrenal failure (e.g. Addison's disease commonly occurring following autoimmune damage to the adrenal gland or TB), or secondary adrenal failure (which occurs due to pituitary failure which may be caused by a pituitary tumour or surgery). In causes of primary adrenal failure ACTH levels from the pituitary will be high and in secondary adrenal failure ACTH levels are inappropriately low. Another common cause of adrenal failure is suppression of the normal pituitary-adrenal axis by steroid therapy such as that used for chemotherapy, rheumatoid arthritis and asthma. Thus, adrenal failure is a relatively common condition and many patients have to take long-term steroid replacement therapy.

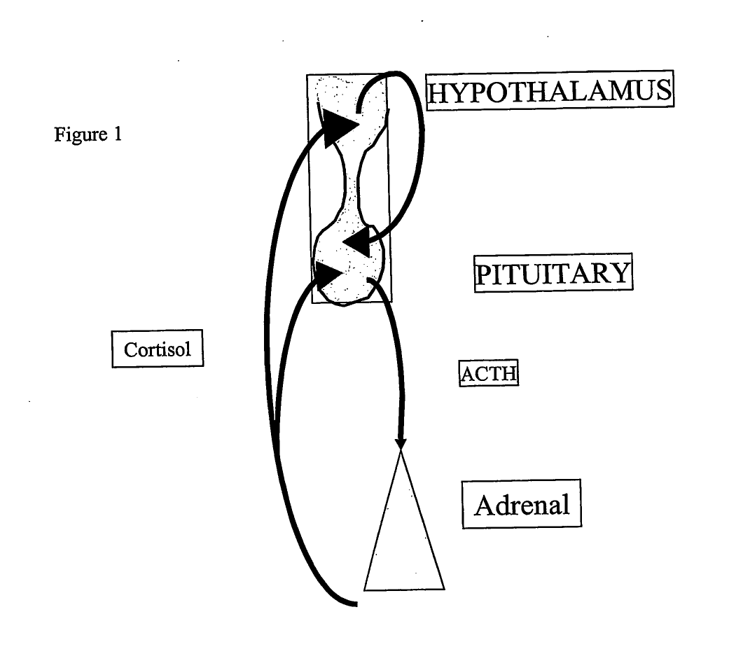

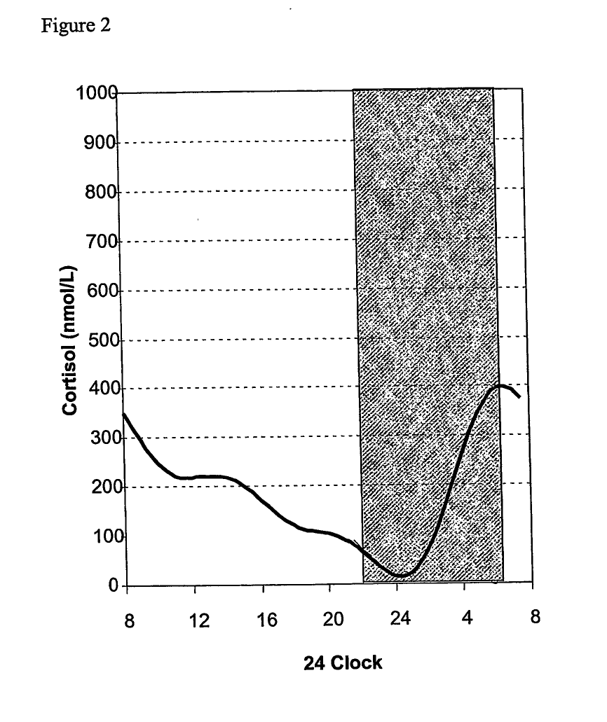

Hydrocortisone is the preferred steroid treatment for patients with adrenal failure. However, other glucocorticoids have been used including cortisone acetate which requires conversion to cortisol in the liver, prednisolone, prednisone, and dexamethasone. Hydrocortisone is the most commonly used drug as it is equivalent to cortisol, is rapidly absorbed and is inexpensive. Cortisol is released from the adrenal gland under the regulation of ACTH derived from the pituitary gland (FIG. 1). There is a circadian rhythm to cortisol release with high levels first thing in the morning and very low levels around midnight (FIG. 2). ACTH and thus cortisol levels begin to rise around 3 am and peak at 7 am gradually falling over the day to a nadir at midnight (Krieger et al., 1971; Ross et al., 1991). Cortisol is a steroid hormone essential for survival especially during stress such as infection. Deficiency in cortisol results in fatigue, wasting, diarrhoea and finally death usually with an Addisonian crisis precipitated by infection.

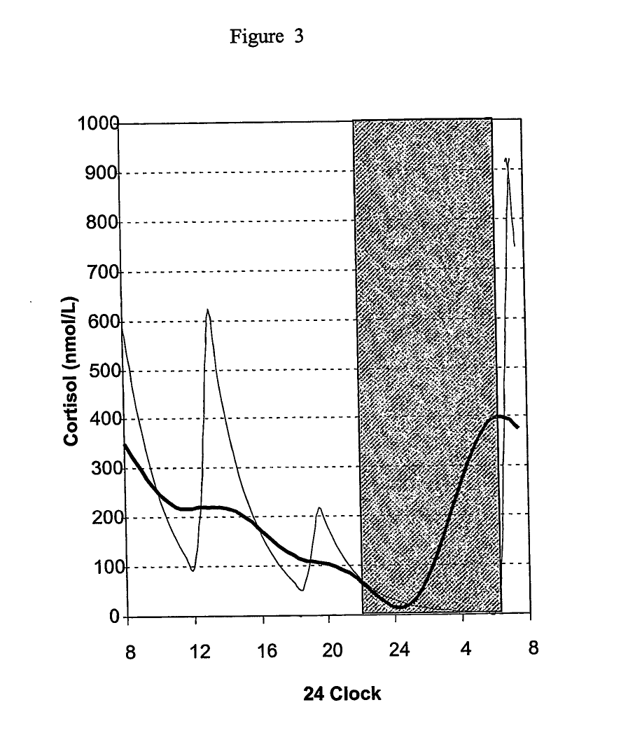

In treating patients with adrenal failure, an attempt is made to mimic the cortisol circadian rhythm by giving a high dose of hydrocortisone when the patient wakes in the morning and then a second dose later in the day. This treatment regimen is effective but does not reflect normal physiology in that patients will wake with undetectable cortisol levels and only get a peak an hour after taking their hydrocortisone (FIG. 3).

The means to regulate controlled drug release are known in the art.

For example, U.S. Pat. No. 4,261,969, which is incorporated by reference, discloses a polymer composition which is enzyme activated. The composition comprises a sensing means which can detect small amounts of a compound in a complex mixture, for example blood, which is an indicator of the body's need for the drug, and a delivery means which senses the change in the sensing means thereby releasing the drug at a required time in a dose dependent manner. The system is suitable for use in the delivery of a contraceptive drug.

A further example is disclosed in EP01077065, which is incorporated by reference. The controlled release formulation comprises a drug core which is surrounded by a release control layer which breaksdown after a predetermined delay. EP01077065 also discloses a drug release layer outside the release control layer which provides for an initial rapid release followed by the release of drugs from the drug core. This provides for the delivery of at least two drug doses in a delayed manner.

U.S. Pat. No. 6,207,197, which is incorporated by reference, discloses a pharmaceutical composition which is adapted to be retained in the stomach to treat diseases, such as ulcers. These are referred to as gastro-retentive drugs. The invention describes microspheres comprising an inner core containing a therapeutic agent surrounded by a water insoluble polymer which is provided with an outer layer of bioadhesive cationic polymer. The adhesive polymer functions to retain the microsphere in the stomach thereby facilitating the concentrated release of the therapeutic agent in the stomach.

EP01053752, which is incorporated by reference, discloses a further example of a preparation which shows controlled release. The preparation comprises two parts, a female and male part, the female part is made from a water insoluble polymer and the male part formed from a composition consisting of ethyl acrylate: methyl methacrylate: trimethylammonioethyl methacrylate co-polymer and a methylacrylic:ethyl acrylate co-polymer. The therapeutic agent is contained within the male and female parts. The formulation is pH sensitive only releasing a therapeutic agent at neutral pH thereby passing through the stomach intact and only releasing in the neutral environment of the small intestine.

WO010957, which is incorporated by reference, discloses an implant which is provided with a coating comprising a polymer matrix which is formed from ethylenically unsaturated monomers which includes a zwitteronic monomer, for example 2-methacryloyloxyethyl-2′-trmethylammoniumethlyphosphate salt. The composition absorbs a therapeutically active substance which is then dried by evaporation of the solvent included with the active substance. The implant is then ready for implantation into the patient and begins to slowly release the active substance.

U.S. Pat. No. 6,217,911, which is incorporated by reference, discloses a controlled release microcapsule for the controlled release of non-steroidal anti-inflammatory drugs for 24 hours to 2 months. The microcapsule is biocompatible and biodegradable and manufactured from DL-lactide-co-glycolide. The composition is topically applied to soft tissues surrounding a surgical incision or wound site. Typically the microspheres are loaded with lidocaine to provide slow release pain relief.

A yet further example of a delayed release formulation is disclosed in WO02/30398, which is incorporated by reference in its entirety. The formulation comprises a core which includes a drug and a disruption agent and further comprises a regulatory membrane coating on the core formed from a mixture of a water soluble gel-forming polymer and a water-insoluble film-forming polymer. The disruption agent is for example an agent which expands on hydration (e.g. hydroxypropylcellulose, sodium starch glycolate, sodium carboxymethylcellulose, croscarmellose sodium or carbomer). The core inlcudes a spheronisation aid (e.g. microcrystalline cellulose). The water soluble gel forming polymer of the regulatory membrane coating is a high viscosity grade hydroxyalkyllcellulose (e.g. hydroxypropylmethylcellulose) or a methyl cellulose. The water insoluble film forming polymer of the regulatory membrane coating is an alkyl cellulose (e.g. ethyl cellulose).

It is apparent that there are means to provide controlled release of therapeutic agents. However the problem addressed by the present invention is how to provide a treatment regime which combines both the delayed and sustained release of a therapeutic agent to provide an optimal treatment regime.

The invention therefore provides a treatment regime suitable for an animal which comprises the administration of at least one therapeutic agent and means which allow for the delayed and sustained release of said therapeutic agent.

According to a first aspect of the invention there is provided a method for the treatment of an animal wherein a therapeutic agent is administered which has the characteristics of controlled release.

In a preferred method of the invention said the delayed and sustained release of said therapeutic agent is in accordance with the circadian rhythm of a patient who is administered said agent.

In a further preferred method of the invention an animal is administered a therapeutic agent which is released in a sustained manner which is followed by a therapeutic agent which is released in a delayed but sustained manner.

Controlled release is construed as, delayed release, sustained release or a combination of delayed and sustained release.

In a preferred method of the invention said method comprises the steps of:
i) providing a combined preparation of a therapeutic agent and a delivery vehicle wherein said vehicle provides for the sustained release of the therapeutic agent;
ii) administering the combined preparation in (i) to an animal requiring treatment such that the therapeutic agent is released in a sustained manner;
iii) providing a combined preparation of a therapeutic agent and a delivery vehicle wherein said vehicle provides for the delayed but sustained release of the therapeutic agent; and
iv) administering the combined preparation in (iii) to an animal requiring treatment such that the therapeutic agent is released in a delayed but sustained manner.

In a preferred method of the invention said animal is human.

In a preferred method of the invention said therapeutic agent is a steroid.

In a further preferred method of the invention said therapeutic agent is cortisol, hydrocortisone, a glucocorticoid or functional derivatives thereof.

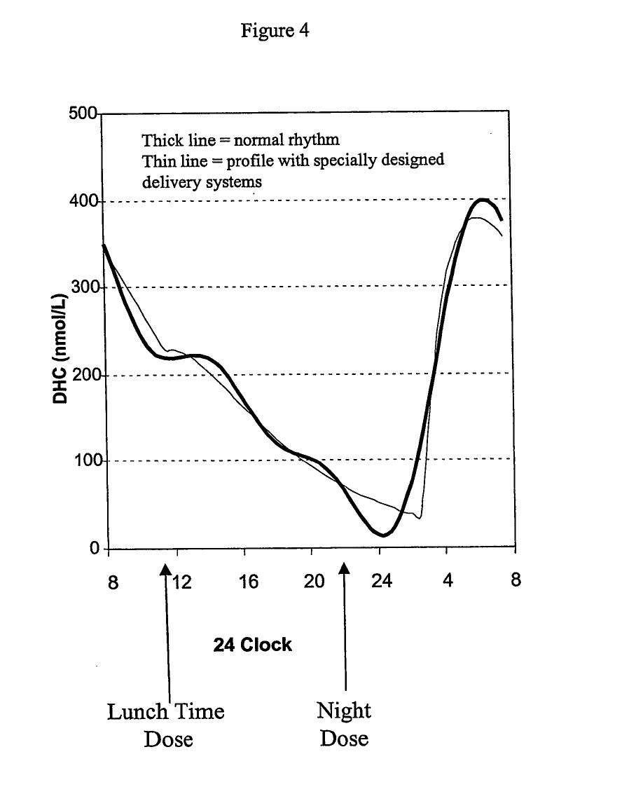

A patient would take a sustained release preparation in the morning and a night-time preparation which would be a delayed and sustained release formulation (FIG. 4). Based on pharmacokinetic modelling this twice daily administration would reproduce the normal circadian rhythm of, for example, cortisol production (FIG. 4).

In a preferred method according to the invention said sustained release preparation is 10-100 times slower than the preparation without the delivery vehicle. Preferably said sustained release is 30-80 times slower and more preferably still about 45-50 times slower than the preparation without the delivery vehicle.

In a further preferred method of the invention said sustained release preparation is administered in the morning, preferably between 08:00 and 12:00.

In a yet further preferred method of the invention said delayed and sustained release formulation is administered in the evening, preferably between 20:00 and 24:00.

According to a further aspect of the invention there is provided a pharmaceutical composition comprising a therapeutic agent and a delivery vehicle characterised in that the delivery vehicle provides for the sustained release of the therapeutic agent.

According to a further aspect of the invention there is provided a pharmaceutical composition comprising, a therapeutic agent and a delivery vehicle characterised in that the delivery vehicle provides for the delayed and sustained release of the therapeutic agent.

In a preferred embodiment of the invention said therapeutic agent is cortisol/hydrocortisone, a glucocorticoid or a functional derivative thereof.

When administered, the pharmaceutical compositions of the present invention are administered in pharmaceutically acceptable preparations. Such preparations may routinely contain pharmaceutically acceptable concentrations of salt, buffering agents, preservatives, compatible carriers, and optionally other therapeutic agents, such as chemotherapeutic agents.

The therapeutic agent of the invention can be administered by any conventional route, including injection. The administration may, for example, be oral, intravenous, intraperitoneal, intramuscular, intracavity, subcutaneous, or transdermal.

The pharmaceutical compositions of the invention are administered in effective amounts. An “effective amount” is that amount of a composition that alone, or together with further doses, produces the desired response. This may involve only slowing the progression of the disease temporarily, although more preferably, it involves halting the progression of the disease permanently. This can be monitored by routine methods or can be monitored according to diagnostic methods.

The pharmaceutical compositions used in the foregoing methods preferably are sterile and contain an effective amount of cortisol/hydrocortisone, glucocorticoids or derivatives thereof for producing the desired response in a unit of weight or volume suitable for administration to a patient. The response can, for example, be monitored by measuring the physiological effects of the composition, such as a decrease of disease symptoms and/or measurement of ACTH levels where appropriate. Assays will be known to one of ordinary skill in the art and can be employed for measuring the level of the response.

The doses of the composition administered to a subject can be chosen in accordance with different parameters, in particular in accordance with the mode of administration used and the state of the subject (ie age, sex, weight, body mass index (BMI). Other factors include the desired period of treatment. In the event that a response in a subject is insufficient at the initial doses applied, higher doses (or effectively higher doses by a different, more localized delivery route) may be employed to the extent that patient tolerance permits.

In general, doses of the composition are formulated and administered in doses between 1 mg and 30 mg, and preferably between 10 mg and 25 mg, according to any standard procedure in the art. More preferably still said sustained release composition is administered between 1 mg and 30 mg at night and between 1 and 15 mg in the morning

An animal as used herein, is a mammal, preferably a human, and including a non-human primate, cow, horse, pig, sheep, goat, dog, cat or rodent.

When administered, the pharmaceutical compositions of the invention are applied in pharmaceutically-acceptable amounts and in pharmaceutically-acceptable compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of cortisol/hydrocortisone or functional derivative thereof, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

The use of a combined therapeutic agent and a delivery vehicle, wherein the delivery vehicle provides for a delayed and sustained release of a steroid for the manufacture of a medicament for use in the treatment of a disease or condition which would benefit from the administration of a steroid.

In a preferred embodiment of the invention said disease or condition is selected from the group consisting of: adrenal dysfunction; rheumatoid arthritis; inflammatory disorders; asthma; nephritis; collagen vascular disorders; connective tissue diseases.

Preferably the adrenal dysfunction is caused by a condition selected from the group consisting of: primary or secondary adrenal failure, congenital adrenal hyperplasia, late-onset congenital adrenal hyperplasia, polycystic ovarian failure.

In a preferred embodiment of the invention adrenal dysfunction is caused by congenital adrenal dysfunction.

Congenital adrenal hyperplasia (CAH) is an autosomal recessive condition commonly due to mutations in the cytochrome P450 21-hydroxylase gene (CYP21) important in the cortisol biosynthetic pathway. Thus, patients have a deficiency in cortisol production, which leads to excess ACTH secretion by the anterior pituitary in an attempt to increase cortisol production (i.e. the loss of cortisol negative feedback at the pituitary, FIG. 1). The rise in ACTH stimulates the adrenal steroid pathway but because there is a block at 21-hydroxylation there is a build up in steroid precursors which are androgenic. This build up in androgenic steroid precursors has important implications for the foetus, infant, child and adult with CAH.

For a female foetus the build up in androgens results in a virilised foetus with ambigious genitalia. In the infant and child the androgens cause pseudo-precocious puberty with excess growth and virilisation. Untreated the child will go through a very early puberty and end up very short. In the adult CAH is associated with infertility, virilisation of the female and steroid deficiency. Treatment of CAH requires steroid replacement therapy both as replacement and to reduce androgenic precursors. Treatment is complex as excessive steroid replacement causes complications in its own right by reducing growth in the child and causing thin bones and skin in the adult.

There are various treatment regimens available which attempt to provide adequate steroid levels during the day and counter the build up of ACTH at night, but none of them provide optimal therapy. The commonest regimen used in children is twice or thrice daily hydrocortisone. This treatment regimen provides supraphysiological levels of hydrocortisone within 1-2 hours of dosing (Charmandari et al., 2001) and doesn't prevent the early morning rise in ACTH and androgenic precursors (Scott et al., 1978; Cutler, 1996). Alternative treatment regimens involve giving a dose of steroids last thing at night which is unphysiological and some patients complain affects their sleeping pattern in addition to which there is a greater risk of giving excess steroid doses as the patient still need steroid replacement during the day.

We propose that the optimal treatment for CAH would be a delayed and sustained release hydrocortisone that could be given last thing at night and reproduce the normal physiological pattern of cortisol secretion.

An embodiment of the invention will now be described by example only and with reference to the following tables, figures and examples:
Table 1 illustrates variability (C.V.) and bias (% difference from healthy matched controls of AUC) for patients on different hydrocortisone regimes;
Table 2 illustrates stepwise multiple linear regression analysis of patient variables with parameters of HC disposition;
Table 3 illustrates suggested hydrocortisone dosing regime;

FIG. 1 represents a schematic representation of cortisol regulation in an animal;

FIG. 2 represents the normal circadian rhythm of cortisol in an animal;

FIG. 3 represents the cortisol profile in a patient taking hydrocortisone three times during a 24 hour period; and

FIG. 4 represents a cortisol profile in a patient taking a sustained and a sustained and delayed release composition of hydrocortisone;

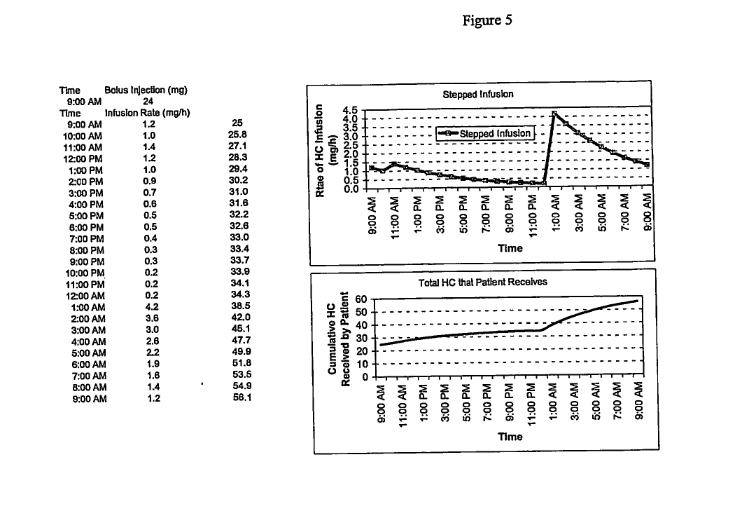

FIG. 5 is a graphical representation of an infusion protocol for the administration of hydrocortisone to a patient suffering from congential adrenal hyperplasis;

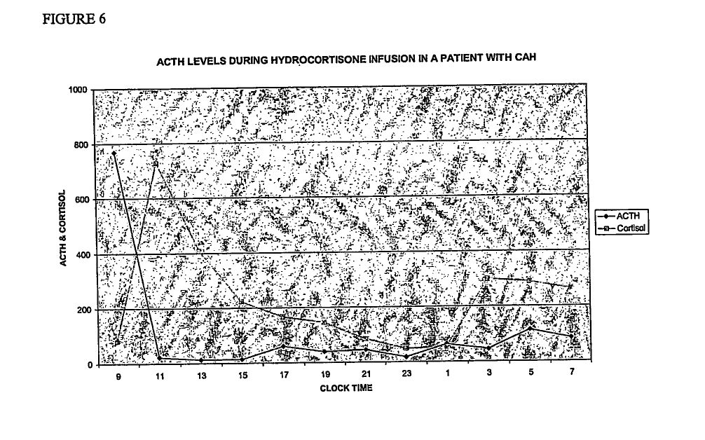

FIG. 6 is a graphical representation of ACTH and cortisol levels in a patient undergoing the infusion protocol described in FIG. 5;

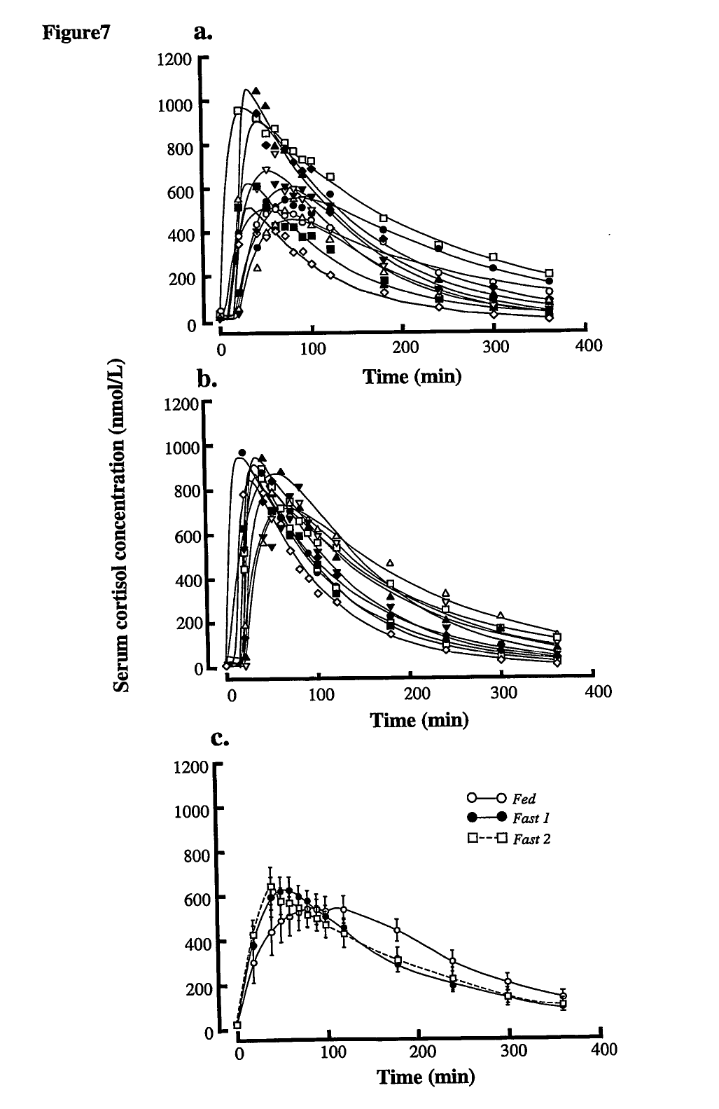

FIG. 7 illustrates serum cortisol concentrations in (a) 10 fasted patients after taking a fixed dose of 10 mg hydrocortisone, (b) 10 fasted patients after taking a weight-adjusted (0.12 mg/kg) dose of hydrocortisone, and (c) following a fixed dose of 10 mg hydrocortisone, in fasting and fed states;

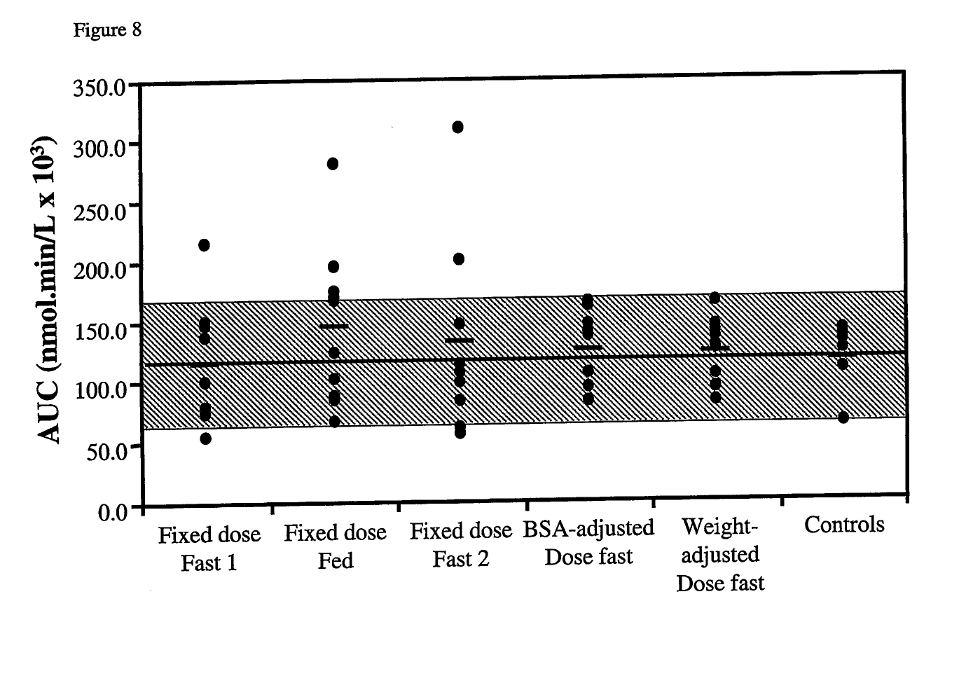

FIG. 8 illustrates a range of AUCs (mean and individual data) according to study groups. The shaded area represents the 95% CI for observations in the healthy control group with the mean as the continuous line;

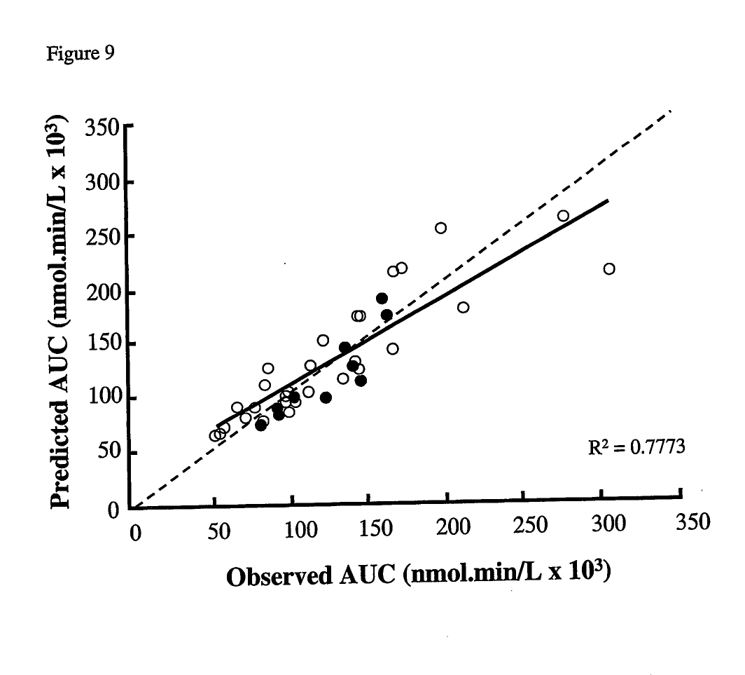

FIG. 9 illustrates predicted AUCs based on 240 min serum cortisol versus observed AUCs (open circles=data from patients in Study 1; closed circles=data from patients in Study 2; bold line=trendline for Study 1 patients which was subsequently used to predict AUCs of the Study 2 patients; dotted line=line of identity); and

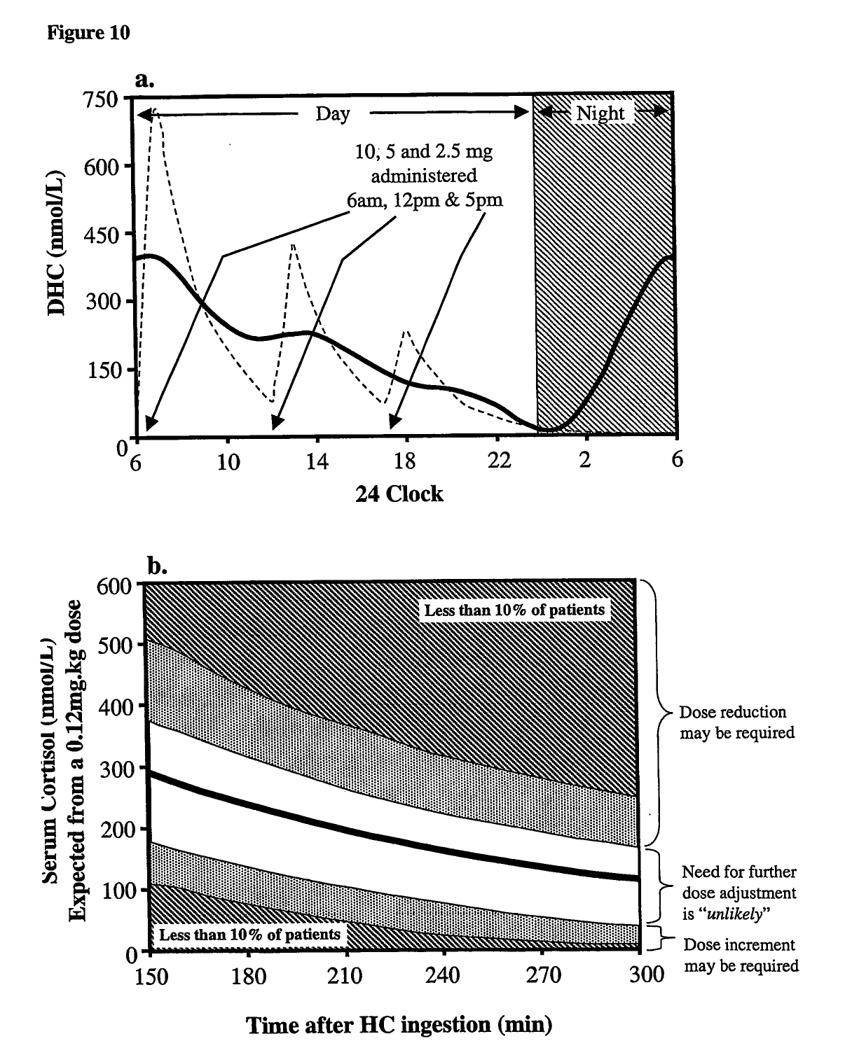

FIG. 10 illustrates (a) Circadian rhythm of serum cortisol in normal subjects from published data (solid line) (10) and simulated cortisol profile for a patient (broken line) following thrice daily hydrocortisone administration according to our optimisation simulation. (b) Nomogram for individual adjustment of hydrocortisone dosage based on serum cortisol estimation 2½h-5 h after a morning HC dose taken on an empty stomach. The lines represent the 10^{th}, 25^{th}, 50^{th}, 75^{th} and 90^{th} centiles.

Materials and Methods

Hydrocortisone Infusion in a Patient with Congenital Adrenal Hyperplasia (CAH)

A treatment regime involves the following protocol. A patient begins a treatment regime at 0830 having taken normal medication the day before. An indwelling cannula is inserted into the patient and basal blood samples are obtained to determine basal levels of cortisol. An infusion cannula is inserted for the administration of a bolus dose of hydrocortisone. Hydrocortisone is administered by infusion once basal samples taken (Patient details: body weight=113 kg; iv bolus 24 mg at 09:00 and follow the infusion rates changed on hourly bases). Total dose for the 1^{st} day is 34.3 mg and 21.8 mg is given by 09:00 in the 2^{nd} day). Samples are taken from the patient periodically from 0900 to 0900 and sampled hourly for cortisol, and ACTH

Hydrocortisone Dosage and Administration

Dilute 100 mg of hydrocortisone in 2 ml of water for injection (concentration 1 mg/20 ul). Give bolus injection of 24 mg hydrocortisone (480 ul or 0.48 ml of hydrocortisone) using a 1 ml insulin syringe. Flush through with normal saline. For infusion put 50 mg of hydrocortisone (1 ml) in 49 mls normal saline=1 mg/ml and the run infusion according to the protocol above.

Assay for Cortisol in Serum Samples

Serum was separated from blood samples immediately after collection and stored at minus 20° C. until analysis. Cortisol concentrations were measured using an ACS:180 automatic chemiluminescence system (Kiron Diagnostic Corporation, East Walpole, USA). Intra- and inter-assay coefficients of variation were less than 7.6%.

### EXAMPLE 1

### Cortisol Metabolism and Clearance

Cortisol secretion under basal non-stressed conditions ranges from 8-25 mg/day (22-69 umol/day) with a mean of 9.2 mg/day (25 umol/day) (de Lacerda et al., 1973; Gallagher et al., 1970). Cortisol although secreted in the unbound state, binds to plasma proteins, cortisol binding globulin (CBG, transcortin) and, to a lesser extent, to albumin (Hammond, 1990). Under basal conditions about 5% to 10% of circulating cortisol is free, about 75% is bound to CBG, and the remainder is bound to albumin. However, it is the free level that is sensed and regulated by the CRH-ACTH axis. Normal CBG has a cortisol binding capacity of 25 ug/dl; increases in total plasma cortisol concentrations above this level result in rapid increases in levels of free cortisol concentration. The cortisol-binding capacity of albumin is greater than that of CBG, but its affinity is lower. Relatively little cortisol is excreted in the urine unchanged—less than 1%. Over 90% of cortisol and the metabolites of cortisol are conjugated in the liver and excreted in the urine. In normal individuals there are circadian fluctuations in the capacity of CBG for cortisol which are lost in patients on chronic replacement. The pharmacokinetics of 20 mg hydrocortisone have been studied after IV and oral administration (Derendorf et al., 1991). After IV administration, hydrocortisone was eliminated with a total body clearance of 18 L/hr and a half-life of 1.7 hr. The volume of distribution was 34 L. Oral bioavailablity averaged 96%. Absorption was rapid, achieving maximum levels after 1 hour.

### EXAMPLE 2

### Modelling a Delayed and Sustained Release Hydrocortisone

Based on published models of circadian cortisol profiles (Chakraborty ey al. 1999) (FIG. 4) we have defined sustained and delayed release tablets, which will provide physiological hydrocortisone therapy (FIG. 4).

### EXAMPLE 3

### Programmable Intravenous Infusion of Hydrocortisone

Normal individuals are studied to establish that programmable intravenous infusion gives a reproducible cortisol profile. To suppress endogenous cortisol production normal individuals will be given dexamethasone 2 mg at 2200 h prior to the start of the study (Patel et al., 1984). At 2400 h a programmed infusion will be started and the individuals will then have 20 minute sampling for 24 hours. To test that this regimen of hydrocortisone will suppress ACTH into the normal range patients with complete adrenal failure (undetectable cortisol levels) will undergo the same protocol without the administration of dexamethasone and the measurement of ACTH.

### EXAMPLE 4

### Sustained/Delayed Release of Hydrocortisone

Means to provide for the sustained release of a therapeutic agent include, by example and not by way of limitation, changing the dissolution rate of for example, hydrocortisone, using available methodologies that incorporate dissolution modifying polymers into the formulation of the delivery system such that a desired rate of release is achieved. These may include fatty acids with different number of carbons, carbohydrates, and derivatives of cellulose.

Delayed release can be obtained by a variety of available methods, which may include the following examples. A hard impermeable capsule which is sealed at the neck edge with hydrogel plug. On ingestion, the capsule becomes exposed to gastric fluids and the water-soluble gelatin cap dissolves, allowing the hydrogel plug to hydrate. At a predetermined and controlled time point after ingestion, the swollen plug is ejected from the capsule body, thereby enabling drug formulation to be released with time of ejection controlled by the length of hydrogel plug and its position relative to the neck of the body.

A further example includes the use of a multilayer capsule/tablet/particulate system wherein three different polymeric layers control the time of release; an inner layer consisting of cathionic polymer dissolving in acidic fluid, a water-soluble intermediate layer, and an outer layer consisting of enteric materials dissolving at pH>5. After ingestion of capsule, drug release can be completely prevented in the stomach due to the acid resistance of the outer polymeric layer. After gastric emptying, the outer layer and the intermediate layer quickly dissolve but the inner polymeric layer still remains and effectively prevents the drug release in the intestine. When the inner polymeric layer is finally dissolved by the acidic fluid the drug content is released. The onset of the drug release, therefore, can be controlled by the thickness of the inner polymeric layer.

A yet further example includes a so-called time control explosion system, consisting of 4 separate layers of seed, drug layer, swelling agent layer and water insoluble membrane. In this system, a rapid drug release is initiated by destruction of outer membrane. The lag time is precisely programmed by changing the outer membrane thickness. As the destruction of the outer membrane is caused by the water uptake of the swelling agent.

A further system comprises a swellable core material, the core being surrounded by an inner coat of a water-insoluble or relatively water-insoluble coating material in which particulate water-insoluble material is embedded. The inner coat is additionally surrounded by an outer coat that contains additional amounts of the desired agent. When the delivery device enters the gastrointestinal tract, the outer coat releases the desired agent contained therein and disintegrates, exposing the inner coat. The particulate matter in the inner coat takes up liquid, thus forming channels interconnecting the drug-containing core with the outside of the delivery device. Through these channels liquid enters the core which then swells to the point at which the inner coat is broken. When the integrity of the inner coat is destroyed, the core then disintegrates, immediately releasing all or most of the drug at a specific site. By controlling parameters in the device, such as the core material, carrier material in the coating, and particulate matter, the location of release of both pulses of the drug can be carefully controlled.

### EXAMPLE 5

### Hydrocortisone Infusion in a Patient with Congenital Adrenal Hyperplasia (CAH)

The following experiment was undertaken to demonstrate that a circadian rhythm hydrocortisone infusion simulating a delayed and sustained release formulation of hydrocortisone can control ACTH in an individual suffering from CAH. The method comprised the infusion of a patient suffering from CAH with hydrocortisone and the measurement of cortisol and ACTH levels. The patient is a man aged 34 years with CAH who is currently treated with oral hydrocortisone.

The infusion protocol for the patient is described in FIG. 5. FIG. 6 shows that in the patient with CAH on conventional therapy the ACTH is very high at 0900 when the cortisol is low. During the infusion the ACTH rapidly falls and the overnight increase in cortisol simulating the delayed and sustained release hydrocortisone prevents the high morning ACTH. Concomitant with the fall in ACTH the 17-OH progesterone fell from >500 nmol/l to a nadir of 67 nmol/l.

### EXAMPLE 6

### Case Studies

The study was carried out in two groups of adrenal-insufficient patients, and a group of healthy subjects. It was approved by the North Sheffield Local Research Ethics Committee, and all participants gave written informed consent. All patients had primary or secondary adrenal insufficiency based on a 0900 h serum cortisol level off treatment of less than 50 nmol/l. Normal controls were matched to the second group of patients for gender, age, height, weight and BSA within 10% of individual patients. Patients on oestrogen replacement were studied after having stopped oestrogen for 6 weeks.

Study 1—Pharmacokinetics of 10 mg fixed dose oral HC: Ten patients (5M, 5F, Age 32-72 yrs, BMI 21.7-35.8 kg/m²) attended the Programmed Investigation Unit on 3 occasions. They discontinued HC replacement from noon on the day prior to the study and fasted from midnight. On the first and third occasions, the patients fasted throughout the study and, on the second occasion, they received a standard breakfast 20-30 minutes before receiving HC. On each of the 3 study days, patients received 10 mg of HC (Hydrocortone™, Merck, Sharp and Dohme) orally, between 0800-0900 h. Peripheral venous blood samples were taken immediately before dosage and at 20, 40, 50, 60, 70, 80, 90, 100, 120, 180, 240, 300 and 360 minutes after dosage; the serum was collected and stored pending assay for HC.

Study 2—Pharmacokinetics of individually tailored dose of oral HC and testing of monitoring protocol: A further 10 patients (6M, 4F; 46-68 y; BMI 24.4-35.5 kg/m²) were studied after receiving HC doses of 5.5 mg/m² BSA (to the nearest practical dose unit) at 0830 h, 1 h before breakfast. The dosage was derived from analysis of the Study 1 data to minimise inter-subject variability. A tablet cutter was used to adjust the HC dose in 2.5 mg quanta, i.e. quarters of 10 mg tablets. Thus, the absolute dose ranged from 7.5 to 12.5 mg. Blood sampling was as in Study 1. Serum concentrations of endogenous cortisol were measured in 7 healthy subjects (3M, 4F; 44-68 y; BMI 23.2-28.7 kg/m²) at the same time points as the patients but without ingestion of HC.

Initial analysis of study 2 showed that if the dose had been adjusted for weight (0.12 mg/kg), the variability for Cₘₐₓ and AUC could have been reduced further. Owing to the discrete nature of dose increments (multiples of 2.5 mg) 9 of the 10 patients who had taken the BSA-adjusted HC dose would still require the same dose if this had been weight-adjusted. Only one of the 10 patients needed a reduction in the HC dose from 12.5 mg to 10.0 mg to be in the nearest weight-adjusted dose range.

Therefore, in the part of our analysis that was related to body weight-adjusted doses, the serum cortisol levels of this patient were normalised to a 10.0 mg dose assuming concentration-dose proportionality.

Kinetic analysis of the serum cortisol concentration-time profiles in the patients was carried out using the software package, P-Pharm (version 1.5, InnaPhase, France), assuming mono-exponential disposition, first-order absorption, and a baseline correction to account for any endogenous levels of cortisol. Values of oral clearance (CL/F), volume of distribution (V/F), absorption rate constant (ka) and absorption lag time (t_{lag}) were obtained from the model fitting and were used to derive other parameter values. Bias in dosing was assessed as the percentage difference in AUC values in patients compared to the AUC of endogenous cortisol in the healthy subjects over the same time period. Variability in the features of the serum cortisol concentration-time profile in the patients was expressed as coefficient of variation (CV). The effect of food intake in Study 1 was assessed using ANOVA, accounting for repeat individual measurements in the fasting state. Body surface area, weight, height, BMI, dose of HC, gender and age were also investigated as co-variables. All statistical analyses were carried out using SPSS version 10.0 (SPSS Inc, USA).

To assess the optimal dosage regimen of HC in patients with adrenal insufficiency, simulations were carried out using the Solver tool in Microsoft Excel™, with the aim of minimizing the residual sum of squares between the simulated 24 h serum HC profile and the measured 24 h profiles reported in the literature (Chachraborty et al 1999). Constraints on the simulated dosage regimen were a maximum of 3 doses between 0600 h and 2200 h and a minimal 2.5 mg increment of HC dosage (compatible with quartering of the available 10 mg tablet).

Based on the results of Studies 1 and 2, Monte Carlo simulations were carried out in virtual patients (n=1000; 32-72 y), using demographic information available for the UK population (Prescott-Clarke, 1996) to construct a nomogram showing the likelihood of the expected serum cortisol concentration at 2.5-5 h after early morning intake of HC. Mathcad (Ver. 6; MathSoft, Edinburgh) was used to generate random numbers and to run the simulations.

### EXAMPLE 7

### Variables Affecting Cortisol Kinetics

There was considerable variability in Cₘₐₓ and AUC values in patients receiving a fixed 10 mg oral dose of HC taken in the fasted state (FIG. 7a; Table 1). This variability was significantly decreased when a BSA- or weight-adjusted dose of HC was assumed (FIG. 7b; Table 1). Weight, height and BSA were major variables affecting HC kinetics; and HC dose also made a significant contribution (Table 2). Of these co-variates, weight had the greatest effect on HC clearance. Compared to inter-subject variability, within-subject variability was low, and the two mean cortisol profiles measured in the fasted state were superimposable (FIG. 7c). Food intake prior to HC ingestion prolonged its absorption half-life (mean±S.E.M. fed vs fast, 43±12 vs 15±4 min; p=0.002) and decreased the oral clearance (0.226±0.031 vs 0.270±0.027 L/min, p=0.05). There was an increase in the variability in Cₘₐₓ (36 vs 31%,) and reduction in the variability in AUC (45 vs 50%) when HC was taken in the fed compared to the fasted state although these changes were not statistically significant.

HC Exposure in Patients Relative to Controls

The AUC of exogenous HC in patients compared to the endogenous AUC in healthy control subjects indicated the least bias when dosage was adjusted for weight (FIG. 8; Table 1).

Single Point Prediction of AUC

Correlation coefficients between the observed AUC values in Study 1 patients and those predicted from their serum cortisol concentrations at specific times were: 240 min (r=0.885, p<0.001), 40+240 min (r=0.912, p<0.001), 60+180+240 min (r=0.950, p<0.001), and 40+60+180+240 min (r=0.950, p<0.001). Thus, 78% of the variability in AUC was explained by the serum cortisol concentration at 240 min. The predicted AUC based on this sample was concordant with observed values in both the original set (30 samples from 10 patients in Study 1) and in the independent group of 10 patients in Study 2 (FIG. 9).

Simulation of Cortisol Dosage Regimens

The serum cortisol profiles after HC administration in studies 1 & 2 were compared to the mean profile of endogenous cortisol in our control subjects. The effects of using different fixed doses (7.5-12.5 mg) were simulated using the observations on the 10 mg dose. Treatment with a 10 mg fixed dose of HC in the fasting state was associated with a 6.3% over-exposure of patients to cortisol compared to our group of controls, and this was greater still in the fed state (24.5%). Individualisation of the dose by weight-adjustment (0.12 mg/kg) resulted in a much smaller bias (4.6%) (Table 1).

The data used for the above analysis refers to the first morning dose of HC. The total daily dose and regimen of administration was estimated using 24 cortisol profiles reported in the literature (Chachraborty et al 1999) and the pharmacokinetic parameters defined by our study. The results show that thrice daily administration given on a dose by weight basis provides optimal replacement within the constraints of the current HC formulation (Table 3 and FIG. 10a).

Nomogram to Adjust Dosage Based on Serum Cortisol Concentration

The predicted population distribution of the serum cortisol concentration-time profile from 150 to 300 min after a 0.12 mg/kg dose of HC is shown in FIG. 10b. This forms the basis of a nomogram to adjust individual HC dosage using a single serum sample. Thus, a cortisol level falling within the 25^{th}-75^{th} centiles suggests that the HC dose taken by the patient results in the predicted cortisol profile (AUC).

Therefore, the dose is appropriate for the patient. If a cortisol level falls above the 75^{th} centile, the HC dose may need to be reduced, whereas a cortisol level that falls below the 25^{th} centile indicates that the patient may require further investigation with regard to possible malabsorption, and the HC dose may need to be increased.

We find that body weight is an important variable determining HC clearance and that prescribing a fixed dose to all patients result in greater under- and over-treatment (bias) compared to a dose adjusted by patient's weight (bias of <5%). Weight-adjusted HC dosage (0.12 mg/kg for the morning dose) produces serum cortisol profiles with less inter-patient variability and is associated with AUC values similar to the endogenous cortisol levels of matched controls during the same study period (0800 h-1400 h). Characterisation of full serum cortisol profiles after a single HC dose is done in some clinics (Feek et al 1981). However, this approach is expensive and time consuming. Based on our data, a single measurement of cortisol at 240 min following HC ingestion can reliably predict the cortisol AUC.

We have modelled HC replacement regimens to provide cortisol profiles as close to physiological levels as practical within the constraints of the current HC preparation (FIG. 4; Table 3). On this basis, we recommend a thrice-daily treatment regimen. The total daily dose (15 mg HC) for a 70 kg patient (175 cm height) is approximately 8.1 mg/m²/day, which is similar to the estimated cortisol production rate (Linder et al 1990). Clinicians generally give a higher replacement dose assuming incomplete absorption and first pass hepatic metabolism (Ten et al 2001). However, the absorption of HC is rapid and oral bioavailability is almost complete (94-96%) (Derendorf et al 1991; Charmandri et al 2001). Our proposed treatment regimen is based on pharmacokinetic studies performed using an early morning dose of HC. Although HC bioavailability in the evening is likely to be the same, clearance may be lower at this time (Charmandri et al 2001). Our total daily and individual HC doses are lower than those currently used by many clinicians on the assumption that HC replacement therapy should be an attempt to replicate normal physiology. It is possible that, because current preparations of HC do not allow normal physiological replacement (cortisol levels are low first thing in the morning), that patients require supra-physiological levels of cortisol at other times of the day. We suggest that changes in HC replacement require careful monitoring including specific questioning about fatigue.

The primary site of cortisol metabolism is the liver (Gower et al 1984). The high oral bioavailability of HC indicates that it has a low hepatic extraction ratio (i.e. the fraction of the dose escaping first-pass hepatic clearance is high) and, for this reason, hepatic enzyme activity is not an important determinant of oral bioavailability. We found that both weight and BSA can predict HC clearance. As predictions using weight were better, and as using weight for dose adjustment in the clinic is easier, we recommend treatment regimens based on weight. The age range for our patients was 32-72 years, and within this range we did not find any additional effects of age on HC pharmacokinetics apart from those determined by weight or BSA. It has been reported that cortisol clearance and volume of distribution increase in parallel at puberty leaving cortisol half-life relatively unaffected (Charmandari et al, 2001). Age is associated with changes in cortisol profiles in response to stress, but mean and integrated cortisol levels are similar in young and elderly normal subjects (Bergendahl et al 2000).

Our results demonstrate that food delays the absorption of HC and increases the variability. This is consistent with previous reports (Barbhaiya et al 1982). In practice, patients are usually encouraged to take their first dose of HC on waking in the fasted state, whereas cortisol profiles are measured later in the morning, often in the fed state. We recommend that patients take their HC on waking, note the time, have breakfast then provide a blood sample 3-5 hours later for monitoring of HC handling according to the nomogram shown in FIG. 10b. Additional dosage adjustment, apart from that of weight adjustment, can be applied accordingly. Optimal HC replacement would be best provided by the development of a delayed release preparation as suggested for the treatment of congenital adrenal hyperplasia (Cutler 1996), and attention should now be given to the development of such a preparation.

TABLE 1

| | | Variability | |
|---|---|---|---|
| | Variability in | in | |
| HC Regime | Cmax | AUC | Bias (AUC) |
| Fixed 10 mg, fasted | 31% | 50% | 6.3% |
| Fixed 10 mg, fed | 36% | 45% | 24.5% |
| BSA-adjusted (5.5 mg/m²), | 10%* | 24%** | 7.4% |
| fasted | | | |
| Weight-adjusted (0.12 mg/kg), | 7%* | 22%** | 4.6% |
| fasted¹. | | | |

¹If weight adjustment was assumed
*p < 0.001 vs fixed dose
**p < 0.05 vs fixed dose

TABLE 2

| | | p value of each | |
|---|---|---|---|
| Parameter | Covariate | covariate | r² |
| HC Clearance* | Weight | <0.001 | 0.380 |
| | Dose | 0.042 | |
| Oral Volume of | Height | <0.001 | 0.421 |
| Distribution | Dose | 0.033 | |
| Cmax^{#} | BSA | <0.001 | 0.604 |
| | Dose | <0.001 | |

*HC = Hydrocortisone
^{#}Cmax = Peak serum cortisol level

TABLE 3

| Patient Weight | Total dose per | 1^{st} dose | | |
|---|---|---|---|---|
| (kg) | day (mg) | (mg) | 2^{nd} dose (mg) | 3^{rd} dose (mg) |
| 50 | 10.0 | 5.0 | 2.5 | 2.5 |
| 55-70 | 15.0 | 7.5 | 5.0 | 2.5 |
| 75-85 | 17.5 | 10.0 | 5.0 | 2.5 |
| 90 | 20.0 | 10.0 | 7.5 | 2.5 |
| 95-110 | 22.5 | 12.5 | 7.5 | 2.5 |
| 115-120 | 25.0 | 15.0 | 7.5 | 2.5 |

### Reference List

CHACHRABORTY, A., KRZYZANSKI, W., JUSKO, W. J. (1999) Mathematical modelling of circadian cortisolconcentrattions using indirect response models: comparison of several models. Journal of pharmacokinetics and Biophartmaceutics, 27,23-43
CHARMANDARI, E., HINDMARSH, P. C., JOHNSTON, A. & BROOK, C. G. (2001) Congenital adrenal hyperplasia due to 21-hydroxylase deficiency: alterations in cortisol pharmacokinetics at puberty. Journal of Clinical Endocrinology & Metabolism, 86, 2701-2708.
CUTLER, G. B. (1996) Treatment of congenital adrenal hyperplasia. Journal of Clinical Endocrinology & Metabolism, 81, 3185-3186.
DE LACERDA, L., KOWARSKI, A. & MIGEON, C. J. (1973) Diurnal variation of the metabolic clearance rate of cortisol. Effect on measurement of cortisol production rate. Journal of Clinical Endocrinology & Metabolism, 36, 1043-1049.
DERENDORF, H., MOLLMANN, H., BARTH, J., MOLLMANN, C., TUNN, S. & KRIEG, M. (1991) Pharmacokinetics and oral bioavailability of hydrocortisone. Journal of Clinical Pharmacology, 31, 473-476.
GALLAGHER, T. F., FUKUSHIMA, D. K. & HELLMAN, L. (1970) The clarification of discrepancies in cortisol secretion rate. Journal of Clinical Endocrinology & Metabolism, 31, 625-631.
HAMMOND, G. L. (1990) Molecular properties of corticosteroid binding globulin and the sex-steroid binding proteins. [Review] [150 refs]. Endocrine Reviews, 11, 65-79.
KRIEGER, D. T., ALLEN, W., RIZZO, F. & KRIEGER, H. P. (1971) Characterization of the normal temporal pattern of plasma corticosteroid levels. Journal of Clinical Endocrinology & Metabolism, 32, 266-284.
PATEL, R. B., ROGGE, M. C., SELEN, A., GOEHL, T. J., SHAH, V. P., PRASAD, V. K. & WELLING, P. G. (1984) Bioavailability of hydrocortisone from commercial 20-mg tablets. Journal of Pharmaceutical Sciences, 73, 964-966.
ROSS, R. J., MIELL, J. P., HOLLY, J. M., MAHESHWARI, H., NORMAN, M., ABDULLA, A. F. & BUCHANAN, C. R. (1991) Levels of GH binding activity, IGFBP-1, insulin, blood glucose and cortisol in intensive care patients. Clinical Endocrinology, 35, 361-367.
SCOTT, R. S., DONALD, R. A. & ESPINER, E. A. (1978) Plasma ACTH and cortisol profiles in Addisonian patients receiving conventional substitution therapy. Clinical Endocrinology, 9, 571-576.
WICHERS, M., SPRINGER, W., BIDLINGMAIER, F. & KLINGMULLER, D. (1999) The influence of hydrocortisone substitution on the quality of life and parameters of bone metabolism in patients with secondary hypocortisolism. Clinical Endocrinology, 50, 759-765.
PRESCOTT-CLARKE P, PRIMATESTA P. Health Survey for England'96. London: The Stationary Office, 1996.
FEEK CM, RATCLIFFE JG, SETH J, GRAY CE, TOFT AD, IRVINE WJ. Patterns of plasma cortisol and ACTH concentrations in patients with Addison's disease treated with conventional corticosteroid replacement. Clinical Endocrinology 1981; 14(5):451-458.
TEN S, NEW M, MACLAREN N. Addison's disease 2001. Journal of Clinical Endocrinology & Metabolism 2001; 86:2909-2922.
LINDER BL, ESTEBAN NV, YERGEY AL, WINTERER JC, LORIAUX DL, CASSORLA F. Cortisol production rate in childhood and adolescence. Journal of Pediatrics 1990; 117(6):892-896.
BARBHAIYA RH, WELLING PG. Influence of food on the absorption of hydrocortisone from the gastrointestinal tract. Drug-Nutrient Interactions 1982; 1(2):103-112.
CHARMANDARI E, JOHNSTON A, BROOK CG, HINDMARSH PC.

Bioavailability of oral hydrocortisone in patients with congenital adrenal hyperplasia due to 21-hydroxylase deficiency. Journal of Endocrinology 2001; 169(1):65-70.
BERGENDAHL M, IRANMANESH A, MULLIGAN T, VELDHUIS JD. Impact of age on cortisol secretory dynamics basally and as driven by nutrient-withdrawal stress. Journal of Clinical Endocrinology & Metabolism 2000; 85(6):2203-2214

1-25. (cancelled)

26. A method of treating adrenal dysfunction in a patient, said method comprising administering to said patient a pharmaceutically effective amount of a combined glucocorticoid and a delivery vehicle, wherein said delivery vehicle provides for delayed and sustained release of said glucorticoid.

27. A method according to claim 26, wherein said glucocorticoid is selected from the group consisting of hydrocortisone, cortisol, cortisone acetate, prednisolone, prednisone, and dexamethasone.

28. A method according to claim 27, wherein said glucocorticoid is hydrocortisone.

29. A method according to claim 26, wherein the adrenal dysfunction is caused by a condition selected from the group consisting of primary and secondary adrenal failure, congenital adrenal hyperplasia, late-onset congenital adrenal hyperplasia, and polycystic ovarian failure.

30. A method according to claim 26, wherein the adrenal dysfunction is a congenital adrenal dysfunction.

31. A method for administering a glucorticoid to an animal, said method comprising the steps of:
i) providing a combined preparation of a glucorticoid and a delivery vehicle wherein said vehicle provides for the sustained release of said glucorticoid;
ii) administering the combined preparation from (i) to an animal requiring treatment such that the glucorticoid is released in a sustained manner;
iii) providing a combined preparation of a glucorticoid and a delivery vehicle wherein said vehicle provides for the delayed and sustained release of the glucorticoid; and
iv) administering the combined preparation from (iii) to an animal requiring treatment such that the glucorticoid is released in a delayed and sustained manner.

32. A method according to claim 31, wherein said animal is a human.

33. A method according to claim 31, wherein said glucocorticoid is selected from the group consisting of hydrocortisone, cortisol, cortisone acetate, prednisolone, prednisone, and dexamethasone.

34. A method according to claim 33, wherein said glucocorticoid is hydrocortisone.

35. A method according to claim 31, wherein said sustained release preparation is about 10-100 times slower than an otherwise identical preparation without the delivery vehicle.

36. A method according to claim 35, wherein said sustained release preparation is about 30-80 times slower than an otherwise identical preparation without the delivery vehicle.

37. A method according to claim 36, wherein said sustained release preparation is about 45-50 times slower than an otherwise identical preparation without the delivery vehicle.

38. A method according to claim 31, wherein the sustained release preparation is administered in the morning, between 8:00 am and 12:00 noon.

39. A method according to claim 31, wherein said delayed and sustained release preparation is administered between 8:00 pm and 12:00 midnight.

40. A method of treating congenital adrenal hyperplasia in a patient, said method comprising administering to said patient a pharmaceutically effective amount of a combined preparation comprising cortisol and a delivery vehicle, wherein said delivery vehicle provides for delayed and sustained release of the cortisol.

41. A method of treating Addison's disease in a patient, said method comprising administering to said patient a pharmaceutically effective amount of a combined preparation comprising cortisol and a delivery vehicle, wherein said delivery vehicle provides for delayed and sustained release of the cortisol.
